SEQUENCE LISTING (1 sequence)

SEQ ID NO: 1 - protein, 6 aa, Bombyx mori
GSGAGA

(19) United States
(12) Patent Application Publication
(10) Pub. No.: US 2019/0159991 A1
(43) Pub. Date: May 30, 2019

(54) SKIN CARE PRODUCTS AND USES THEREOF

(71) Applicant: Jonas Wang, Baldwin Park, CA (US)

(72) Inventor: Jonas Wang, Baldwin Park, CA (US)

(73) Assignee: Wang, Jonas

(21) Appl. No.: 16/320,875

(22) PCT Filed: Jul. 25, 2017
(86) PCT No.: PCT/US2017/043630

§ 371 Date: Jan. 25, 2019

Related U.S. Application Data
(60) Provisional application No. 62/367,820, filed on Jul. 28, 2016.

(51) Int. Cl. A61K 8/67; A61K 8/02; A61Q 19/08

(52) U.S. Cl. CPC A61K 8/671; A61K 8/676; A61K 8/678; A61K 2800/592; A61Q 19/08; A61K 2800/524; A61K 8/0212

(57) ABSTRACT

This invention relates to a skin care formulation containing, among others, Vitamins A, C, and E, and derivatives thereof and to a skin care product where the formulation is impregnated in a delivery system, which can be made of pure heavy-metals-free silk membrane or mask, to retard the effects of aging of the skin and generally improve the quality of the skin, particularly human facial skin.

### CROSS REFERENCE TO RELATED APPLICATION

This application claims priority to U.S. Provisional Application No. 62/367,820 filed on Jul. 28, 2016. The content of the application is incorporated herein by reference in its entirety.

### FIELD OF THE INVENTION

This invention relates to a therapeutic skin care formulation containing, among others, Vitamins A, C, and E, and derivatives or salts thereof and to a skin care product where the formulation is impregnated in a delivery system, which can be made of pure heavy-metals-free silk membrane or mask to retard the effects of aging of the skin and generally improve the quality of the skin, particularly human facial skin.

### BACKGROUND OF THE INVENTION

Many skin care products currently available to consumers are directed primarily to improving the health and/or physical appearance of the skin. Among these skin care products, many are directed to delaying, minimizing or even eliminating skin wrinkling and other histological changes typically associated with the aging of skin or environmental damage to human facial skin. Recently, facial masks in Asia especially in China, Taiwan, Korea and Japan has been the major product for facial cosmetic e.g., in Taiwan and China over 2 billion pieces of facial masks are sold every year and over 30% of the facial beauty market is on the facial masks products. However, most of the facial mask products are mainly made of non-woven fabric which does not adhere to the skin and dried out quickly, do not provide enough moisture to keep the skin hydration, and ineffective in deliver active ingredients to the basal layers of skin. Although there are some facial masks made of micro-bio-cellulose which can provide enough moisture to keep the skin hydration for a longer period of time, however, it is ineffective in deliver active ingredients to the basal layers of skin. Therefore, there is a need for skin care products such as a facial mask which not only adhere and biocompatible to the skin, maintain the moisturization and hydration of the skin for a longer period of time, but also effectively deliver active skin ingredients to the basal layers of the skin to enhance the growth of stem cells.

### SUMMARY OF INVENTION

This invention relates to skin care products and uses thereof.

In one aspect, the present invention relates to a skin care composition, comprising:

(a) from about 70% to 80% of purified water;

(b) from about 0.0005% to about 1%, such as 0.001% to about 1%, more preferably from about 0.005% to about 0.75%, even more preferably from about 0.01% to about 0.6%, and still more preferably from about 0.02% to about 0.4%, most preferably from about 0.04% to about 0.1%, of the Vitamin A alcohol, retinal or retinyl palmitate or the derivatives or salts thereof;

(c) from about 1% to about 15%, more preferably from about 1.5 to about 10%, even more preferably from about 2% to about 8%, and still more preferably from about 3% to about 6%, most preferably from about 4% to about 5%, of Vitamin C or the derivatives or salts thereof;

(d) from about 0.005% to about 0.5%, such as 0.01% to about 0.5%, more preferably from about 0.01% to about 0.4%, even more preferably from about 0.02% to about 0.3%, and most preferably from about 0.03% to about 0.2%, of Vitamin E or the derivatives or salts thereof and a dermatologically or cosmetically acceptable carrier. Preferably, Vitamins E, A, and C (or the derivatives or salts thereof) are present in a ratio of about 1:1-2:100-150, such as 1:1:125.

The composition can further comprise one or more of the following:

(e) from about 0.05% to about 8% more preferably from about 0.1% to about 7%, even more preferably from about 0.5% to about 6%, and most preferably from about 3% to about 5%, of a moisturizing agent(s), e.g., one or more selected from the group consisting of Glycerin, Sodium Hyaluronate, Snail extract, Mannitol, Panthenol, Cyclopeptide, Ammonium Glycyrrhizate and a combination thereof;

(f) from about 1% to about 3%, more preferably from about 1.2% to about 2.8%, even more preferably from about 1.5% to about 2.5%, and most preferably from about 1.8% to about 2.0%, of a structuring agent, e.g., one or more selected from the group consisting of Xanthan Gum and Chondrus Crispus Extract, Hydroxy Ethyl Cellulose, and combinations thereof;

(g) from about 0.25% to about 2%, more preferably from about 0.3% to about 1.5%, and most preferably from about 0.5% to about 1%, of by weight of the composition of a buffer/pH adjusting agent e.g., one or more selected from the group consisting of citric acid and lactic acid; and

(h) from about 0.1% to about 0.5% more preferably from about 0.15% to about 0.45%, still more preferably from about 0.2% to about 0.4%, most preferably from about 0.25% to about 0.3%, by weight of the composition a preservative, e.g., one or more selected from the group consisting of phenoxyethnol, ethylhexlglycerin, and methyl/propyl paraben.

The formulation comprises an effective combination of Vitamins A, C, and E or the derivatives or salts thereof. In some embodiments, the formulation can further comprise a compound selected from the group consisting of hydroxy acids, desquamatory agents, sunscreens, anti-oxidants, and combinations thereof. The carrier can be various suitable carriers, e.g., the form of essence liquid, a cream or lotion. The essence liquid can be impregnated in a delivery system made of pure gentle mixing double-sided knitting silk membrane.

The invention also provides a delivery system comprising (i) a formulation described above and (ii) a substrate, wherein the formulation is impregnated in or on the substrate. The substrate can comprise a pure gentle mixing double-sided knitting silk membrane. For example, the delivery system can be a facial mask.

Also provided is a method of regulating skin condition, or regulating visible and/or tactile discontinuities in the texture of mammalian skin. The method includes applying to a surface of skin in need thereof (e.g., facial skin) a formulation or a delivery system (e.g., a facial mask) described above.

The details of one or more embodiments of the invention are set forth in the description below. Other features, objectives, and advantages of the invention will be apparent from the description and from the claims.

### BRIEF DESCRIPTION OF THE DRAWING

FIG. 1 is a diagram showing cosmetic beneficial effects of a skin care product (Example 3) of this invention measured by VISTA® Complexion analysis (n=8).

### DETAILED DESCRIPTION OF THE INVENTION

This invention is based, at least in part, on unexpected findings that a topical composition regulates and improves aging skin conditions more effectively than conventional compositions. As mentioned above, there is a need for skin care products such as a facial mask which not only adhere and biocompatible to the skin, maintain the miniaturization and hydration of the skin for a longer period of time, but also effectively deliver active skin ingredients to the basal layers of the skin to enhance the growth of stem cells

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. To many people, skin wrinkles and pigmentation are a reminder of the disappearance of youth. As a result, the elimination of wrinkles and pigmentation has become a booming business in youth-conscious societies. Treatments range from cosmetic creams and moisturizers to various forms of cosmetic surgery.

Extrinsic or intrinsic factors may result in the thinning and general degradation of the skin. For example, as the skin naturally ages, there is a reduction in the cells and blood vessels that supply the skin. There is also a flattening of the dermal-epidermal junction which results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990, which is incorporated by reference herein in its entirety.

### Age Associated Structural Changes

Although many of the effects of the aging of the human skin are the result of underlying structural changes which build up over a period of years and can only be detected histologically prior to middle age, these changes and effects being to appear clinically about middle age, namely between about 35 and 45 years of age, and become more and more evident and pronounced thereafter. The more apparent effects of aging have already been referred to above, and each is associated with one or more underlying structural changes in the skin. For example, blotchiness or mottling (hyperpigmentation) is due to changes in the melanocytes in the population of epidermal cells. These pigment producing cells which, unlike the keratinocytes remain at the base of the epidermis lose their normal regulation process with aging and produce excess pigments which cause the blotchiness and mottling.

However, aside from such obvious cosmetic changes in the skin, there are a number of other changes which are more important though less apparent, including loss of sensory acuity and ability to heal wounds, decreased blood flow and decrease in the thickness of the skin. Older people have less sensitivity to pain and a longer response time. Thus, pain due to irritation or injury is not felt as soon or to the same extent as in young people with the result that superficially minor but potentially serious injuries may be sustained without the individual being aware of the injury until serious damage has occurred.

The surface temperature of the skin in older people is somewhat lower than the skin temperature in younger people, so that they often feel cold. This is due to a decrease in the blood supply to the skin due to loss of small blood vessels and decreased proliferation of new capillaries and small blood vessels in the skin. This is at least one of the causes of the loss of sensory acuity and response to pain. Furthermore, the decreased blood supply decreases the rate at which irritants and toxins are cleared from the skin tissue.

Still further, the skin of older people is more easily torn than that of younger people, since both the epidermis and dermis become thinner with age. As a result, there is less bulk to protect underlying organs and therefore more risk of serious injury. Moreover, when wounds or injuries are sustained, healing of the wounds is much slower in older people and may take as much as twice as long to heal as in younger persons.

The underlying causes of the above gross skin effects may be understood more readily from the following discussion of the specific changes in the epidermis and dermis as aging progresses.

### 1. Epidermis

With increasing age and exposure of a human to sun and other environmental traumas, cells divide at a slower rate (decreased capacity to renew themselves). They show marked irregularities in size, shape and staining properties; orderliness (polarity) from below to above is lost. The thickness of the epidermis decreases (atrophy). The horny layer which comprises the barrier against water loss and penetration of chemicals becomes abnormal due to the shedding (exfoliation) of cells in large group or clusters instead of as individual cells, resulting in roughness, scaling and dryness. There is loss of the orderly transformation of living epithelial cells into cornified dead cells which are shed at the surface, that is, differentiation is impaired. Aberrant differentiation results in numerous foci of abnormal epithelial growths or tumors, the most frequent and important of which are actinic keratoses. After many years these can transform into frank skin cancers called basal cell and squamous cell cancers. Pigment producing cells (melanocytes) can also become altered forming flat, dark growths (lentigo melanoma) which may progress to malignant melanoma. The cells which make up these premalignant growths are destroyed by topical Vitamin A alcohol and its derivative or salt.

### 2. Dermis

The cells which make the fibers of the dermis become smaller and sparser with increasing age, usually in sun damaged facial skin. There is a great loss of collagen fibers resulting in looseness and easy stretchability of the skin; elastic fibers become abnormal so that the skin does not promptly snap back after being stretched. Since the fibrous components comprise more than 90% of the bulk of skin of which 95% is collagen, the degradation of these fibers, especially collagen, is mainly responsible for wrinkling, laxness and loss of elasticity.

Small blood vessels become thin walled, dilated and often ruptured. Vascular supply thereby becomes compromised.

### Skin Care Compositions and Related Silk Facial Membranes

As disclosed herein, this invention discloses Vitamins-containing skin care compositions and related silk facial membranes. In some embodiments, the skin care compositions contain a combination of Vitamins A, C and E, or the derivatives or salts thereof, impregnated in a pure silk membrane and effectively enhance skin cell regeneration and improve the quality of the skin, particularly human facial skin. More particularly, the present invention relates to a therapeutic skin care compositions containing Vitamins A, E, and C (or the derivatives or salts thereof) impregnated in a delivery system made of biocompatible/biodegradable pure silk membrane. Various effects of aging of skin due to impairment of differentiation of epidermal epithelial cells and loss of collagen fibers, abnormal changes in elastic fibers and deterioration of small blood vessels in the dermis of the skin are retarded by applying topically to the epidermis in a maintenance therapy program effective amounts of a combination of Vitamins A alcohol or its derivatives or salts, Vitamin C or its derivatives or salts, and Vitamin E or its derivatives or salts impregnated in a delivery system made of pure heavy-metals-free biocompatible/biodegradable silk membrane or mask, such that epithelial growths are substantially reduced and prevented and the skin substantially regains and maintains its firmness, turgor and elasticity. Moreover, with persistent treatment dermal blood cells and vessels increase and the epidermis and dermis thicken, resulting in improved ability of the skin to sense, resist and recover from irritation or injury. Further, hyperpigmentation, lines and wrinkles due to aging are reduced and prevented. The therapeutic skin care Vitamins containing product is particularly useful for human facial skin and preferably applied in amounts insufficient to cause excessive irritation.

Vitamin A is a group of unsaturated nutritional organic compounds that includes retinol, retinal, retinoic acid, and several proVitamin A carotenoids (most notably beta-carotene). Vitamin A such as retinol and its derivatives or salts can provide measurable skin regulating benefits. For example, topical 13-cis-retinoic acid helps to regulate the signs of skin aging, i.e., reduce or efface the visibility of the fine lines, wrinkles, and other forms of uneven or rough surface texture associated with aged or photodamaged skin. Retinol or retinyl palmitate or its derivatives or salts are considered to be the prodrugs of 13-cis-retinoic acid because they will convert to 13-cis-retinoic acid when it reaches the base cell level. These compounds have also been found useful in reducing the overall oiliness of skin.

In formulating products containing retinol, retinyl palmitate and its derivatives or salts, much attention is directed toward providing compositions which deliver and retain optimal concentrations of the active ingredients e.g., retinoic acid in the stratum corneum with minimum absorption into the systemic circulation. Furthermore, promoting user compliance with respect to chronic treatment regimens is also important. Most of the conventional retinol, retinyl palmitate and its derivatives or salts formulations on the market, however, can be drying and irritating. Such formulations may cause individuals to refrain from using retinol and its derivatives products as frequently and copiously as is necessary for optimum benefit.

To address the problem, this invention in one embodiment provides a unique design of a therapeutic skin care mask (e.g., facial mask) for retarding and reversing the loss of collagen fibers, abnormal changes in elastic fibers, the deterioration of small blood vessels, and the formation of abnormal epithelial growths in sun-damaged human skin. Also provided is a skin care method comprising applying topically to the epidermis of skin, e.g., the facial skin, a composition comprising effective combination of Vitamins A, C and E and/or their derivatives or salts in a delivery system made of pure gentle mixing double-sided knitting silk membrane for a program of skin (e.g., facial skin) maintenance therapy, whereby the skin substantially regains and maintains its firmness, turgor and elasticity during said therapy, said combination composition of Vitamins A, C, E and its derivatives or salts being selected so as to provide an irritating-free mask products.

Silk is a natural protein fiber. Silk emitted by the silkworm consists of two main proteins: sericin, being the sticky material surrounding fibroin, while fibroin, being the structural center of the silk. Fibroin is largely made of the amino acids Gly-Ser-Gly-Ala-Gly-Ala (SEQ ID No.: 1) and forms beta pleated sheets, β-keratin. The structure of silk is shown below:

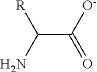

Due to its structure and negative charges, silk can be in a fully hydrated state once applied to skin and adhere to the skin by hydrogen bonding and Van der Waal force to its ultimate intimacy. It is also an ideal delivery system for active skin ingredients such as Vitamin A, C, and E. Silk enhances the bioavailability of Vitamins (A, C, and E) to achieve the optimal clinical efficacy. Silk is highly biocompatible within the body, and can be used a material for medical textiles for, e.g., burn patients, to feel comfortable and contribute towards natural healing property, especially for burn injuries. Silk also demonstrates an impressive range of material properties based on a variety of processing protocols. Therefore, silk is an ideal biomaterial as a delivery system for medical application.

Various silk materials can be used in this invention. Due to its use for skincare, silk materials should be free or substantially free of substances harmful or potentially harmful, such as heavy metals, to the skin and the body. Accordingly, care should be taken to use silk made by silkworms raised in an environment free of such substances (e.g., fed with leaves free of such substances). Alternatively, a process to remove such substances from silk is needed.

This invention discovers that the pure silk may be an ideal candidate for a target delivery system for the Vitamins combination that effectively re-generating the stem cells in skin. In one example, this invention discloses a dual knitting process using polyesters fibers to reinforce a silk protein membrane. Examples of polyesters fibers that can be used including polyester resin, saturated or unsaturated polyester resin. By mimicking the natural roughness and stiffness of skin, this biodegradable/biocompatible structure helps to encourage vigorous skin cell turn over (stem cells growth). While certain biomaterials are at the center of research into skin cells regeneration, none of these existing materials can match the pure silk's biocompatibility and delivery efficacy.

As disclosed herein, it was found that the pure silk membrane is the ideal delivery system to optimize the bioavailability of the Vitamins to achieve its maximum clinical benefits. In a clinical evaluation it was found that compositions containing combination of Vitamins A, C, and E in a pure gentle silk membrane delivered the skin regulating benefits with reduced wrinkles, de-pigmentation, moisturization, smoothness but with no irritation. These compositions have improved user acceptance and, thus, promoted better user compliance with a concomitant overall improvement in skin regulating benefit.

It is, therefore, an object of the present invention is to provide Vitamin A, C, E (or the derivatives or salts thereof) containing facial mask compositions having improved skin compatibility and skin conditions. Another object of the present invention is to provide Vitamins A, C, E (or the derivatives or salts thereof) combination compositions containing in a gentle pure silk membrane which provides skin rejuvenation benefits without irritation or undesirable side effects. Still another object of the present invention is to provide Vitamins A, C, E (or the derivatives or salts thereof) combination compositions in a gentle pure silk membrane which do not substantially impact stability or bioavailability, but offer the maximum biocompatibility to the facial skin. These and other objects of this invention will become apparent in light of the disclosure herein.

### Uses

The formulation and products (e.g., masks) disclosed in the present invention can be used to treat, regulate, or improve skin conditions.

Many adults who have had a good deal of sun exposure in childhood will show the following gross cutaneous alterations in adult life: wrinkling, leatheriness, yellowing, looseness, roughness, dryness, mottling (hyperpigmentation) and various premalignant growths (often subclinical). These changes are most prominent in light-skinned persons who burn easily and tan poorly. The baleful effects of sunlight are cumulative, increasing with time. Although the anatomic degradation of the skin is most advanced in the elderly, the destructive effects of excessive sun exposure are already evident by the second decade. Serious microscopic alterations of the epidermis and dermis occur decades before these become clinically visible. Wrinkling, yellowing, leatheriness, loss of elasticity are very late changes.

In view of the foregoing, it is believed that a combination of Vitamins A, C and E influences ultrastructural and proliferative properties of epidermal cells. However, conventional uses of Vitamins A, C and E combination have generally involved short term treatments in which relatively large doses of the Vitamin A alcohol and its derivatives or salts are applied (i.e. sufficient to cause significant irritation and often peeling) in order to obtain a quick cure or treatment of the particular condition, as opposed to persistent treatment of normal aging skin.

All percentages and ratios used herein are by weight of the total composition and all measurements made are at 25° C., unless otherwise designated.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The term “topical application”, as used herein, means to apply or spread the compositions of the present invention onto the surface of the skin.

The term “dermatologically-acceptable,” as used herein, means that the compositions or components thereof so described are suitable for use in contact with human skin without undue toxicity, incompatibility, instability, allergic response, and the like.

The term “safe and effective amount” as used herein means an amount of a compound or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound judgment of the skilled artisan.

The term “skin compatibility,” as used herein means the ability of skin to tolerate long term application of topical compositions with minimal adverse skin reactions such as stinging, burning, redness, itching and folliculitis.

The compositions of the present invention are useful for topical application and for regulating skin condition, including visible and/or tactile discontinuities in skin (especially the skin surface; such discontinuities are generally undesirable). Such discontinuities may be induced or caused by internal and/or external factors, and include the signs of skin aging described herein. The term “regulating skin condition” includes prophylactically regulating and/or therapeutically regulating skin condition, including visible and/or tactile discontinuities in skin. As used herein, prophylactically regulating skin condition includes delaying, minimizing and/or preventing visible and/or tactile discontinuities in skin. As used herein, therapeutically regulating skin condition includes ameliorating, e.g., diminishing, minimizing and/or effacing, discontinuities in skin. Regulating skin condition involves improving skin appearance and/or feel.

The compositions of the present invention are useful for regulating signs of skin aging, more especially visible and/or tactile discontinuities in skin texture associated with aging. “Regulating the signs of skin aging” includes prophylactically regulating and/or therapeutically regulating one or more of such signs (similarly, regulating a given sign of skin aging, e.g., lines, wrinkles or pores, includes prophylactically regulating and/or therapeutically regulating that sign). As used herein, prophylactically regulating such signs includes delaying, minimizing and/or preventing signs of skin aging. As used herein, therapeutically regulating such signs includes ameliorating, e.g., diminishing, minimizing and/or effacing signs of skin aging.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles, including both fine superficial wrinkles and coarse deep wrinkles, skin lines, crevices, bumps, large pores (e.g., associated with adnexal structures such as sweat gland ducts, sebaceous glands, or hair follicles), scaliness, flakiness and/or other forms of skin unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as malesma, age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

It is to be understood that the present invention is not to be limited to regulation of the above mentioned “signs of skin aging” which arise due to mechanisms associated with skin aging, but is intended to include regulation of said signs irrespective of the mechanism of origin. As used herein, “regulating skin condition” is intended to include regulation of such signs irrespective of the mechanism of origin.

The present invention is especially useful for therapeutically regulating visible and/or tactile discontinuities in mammalian skin texture, including texture discontinuities associated with skin aging. As used herein, therapeutically regulating such discontinuities includes ameliorating, e.g., diminishing, minimizing and/or effacing visible and/or tactile discontinuities in the texture of mammalian skin, to thereby provide improved skin appearance and/or feel, e.g., a smoother, more even appearance and/or feel. Such visible and/or tactile discontinuities in skin texture include crevices, bumps, pores, fine lines, wrinkles, scales, flakes and/or other forms of textural unevenness or roughness associated with skin aging. For example, the length, depth, and/or other dimension of lines and/or wrinkles are decreased, the apparent diameter of pores decreases, or the apparent height of tissue immediately proximate to pore openings approaches that of the interadnexal skin.

The present invention is also especially useful for prophylactically regulating visible and/or tactile discontinuities in mammalian skin texture, including texture discontinuities associated with skin aging. As used herein, prophylactically regulating such discontinuities includes delaying, minimizing and/or preventing visible and/or tactile discontinuities in the texture of mammalian skin, to thereby provide improved skin appearance and/or feel, e.g., a smoother, more even appearance and/or feel.

The present invention relates to the use of effective strength of a combination of Vitamins A, E, and C (or the derivatives or salts thereof) in moderating and preventing the aging changes of the exposed areas of the skin, especially the face. In particular, the methods of the present invention retard the effects of normal aging of the skin due to impairment of the differentiation of epidermal epithelial cells and due to loss of collagen fibers, abnormal changes in the elastic fibers and deterioration of small blood vessels of the dermis of the skin.

The Vitamins A, C, and E (or the derivatives or salts thereof) combination may be applied to the skin in any suitable non-toxic, dermatologically acceptable vehicle, preferably a non-volatile, emollient or lubricating vehicle, in an amount and at a frequency which are insufficient to cause excessive irritation of the skin. Generally, concentration of Vitamin A alcohol and its derivatives or salts in the range of about 0.001 to 1% by weight of the vehicle are preferred.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a range of values which would not be considered by a person of ordinary skill in the art as substantially different from the baseline values. For example, the term “about” may refer to a value that is within 20%, 15%, 10%, 9%, 8%, 7%, 6%, 5%, 4%, 3%, 2%, 1%, 0.5%, 0.1%, 0.05%, or 0.01% of the stated value, as well as values intervening such stated values.

### DETAILED DESCRIPTION OF PREFERRED EMBODIMENTS

The composition of the present invention comprises as a safe and effective amount of Vitamins A, C, and E (or the derivatives or salts thereof) combination formula. From about 0.001% to about 1%, more preferably from about 0.005% to about 0.75%, even more preferably from about 0.01% to about 0.6%, and still more preferably from about 0.02% to about 0.5%, most preferably from about 0.04% to about 0.2%, of the Vitamin A alcohol, retinal or retinyl palmitate; Exemplary derivatives of the foregoing Vitamin A compounds include retinol, retinal, and retinyl palmitate as well as others known in the art.

From about 1% to about 15%, more preferably from about 1.5 to about 10%, even more preferably from about 2% to about 8%, and still more preferably from about 3% to about 6%, most preferably from about 4% to about 5%, of Vitamin C;

Exemplary derivatives of the foregoing Vitamin C compounds include Sodium Ascorbyl Phosphate, Magnesium Ascorbyl Phosphate, Ascorbyl Glucoside, 3-O-Ethyl Ascorbic Acid as well as others known in the art. When used, salts, derivatives, and salt derivatives of ascorbic acid are preferably those having substantially the same efficacy as ascorbic acid in the methods of regulating skin condition described herein.

From about 0.005% to about 0.5%, more preferably from about 0.01% to about 0.4%, even more preferably from about 0.02% to about 0.3%, and most preferably from about 0.03% to about 0.2%, of Vitamin E; and a dermatologically or cosmetically acceptable carrier.

Exemplary derivatives of the foregoing Vitamin E compounds include Vitamin E and Vitamin E palmitate as well as others known in the art.

Preferably, Vitamins A, C, and E are present in a ratio of about 1 to about 50 to about 12.5.

In a preferred embodiment, the Vitamins A, C, E combination contains a limited amount of the salt form and is more preferably substantially free of salts. The Vitamin A, C, and E combination hereof having a pH of from about 4 to about 7 typically contain less than about 50% of the salt form.

The composition of the present invention can comprise various optional components as discussed below.

### Preservative Component

From about 0.1% to about 0.5% more preferably from about 0.15% to about 0.45%, still more preferably from about 0.2% to about 0.4%, most preferably from about 0.25% to about 0.3%, by weight of the composition a preservative, e.g., one or more selected from the group consisting of phenoxyethnol, ethylhexlglycerin, and methyl/propyl paraben. Included the composition of the present invention is a preservative component comprising a phenoxyethanol. The preservative component of the present invention provides substantially no negative impact to the active's stability. The phrase, “substantially no negative impact,” as used herein, means that less than about 10% of the active is chemically degraded or otherwise altered resulting in an inactive degradation product when in the presence of the preservative component in the composition herein during a one month period at a temperature of 40° C.

### Carrier

The compositions of the present invention can comprise from about 1% to about 70-80% of a dermatologically or cosmetically acceptable carrier within which the compositions of the present invention is incorporated to enable Vitamins A, C and E combination and preservative component, as well as other optional actives, to be delivered to the skin at an appropriate concentration.

The carrier may contain one or more dermatologically acceptable solid, semi-solid or liquid fillers, diluents, solvents, extenders and the like. The carrier may be solid, semi-solid or liquid. Preferred carriers are substantially liquid. The carrier can itself be inert or it can possess dermatological benefits of its own. Concentrations of the carrier can vary with the carrier selected and the intended concentrations of the essential and optional components.

Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention. Preferred components of the compositions of this invention should be capable of being comingled in a manner such that there is no interaction which would substantially reduce the efficacy of the composition under ordinary use situations.

The type of carrier utilized in the present invention depends on the type of product form desired for the composition. The topical compositions useful in the subject invention may be made into a wide variety of product forms such as are known in the art. See those listed in the reference list. These include, but are not limited to, lotions, creams, gels, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and liposomes.

Preferred carriers contain a dermatologically acceptable, hydrophilic diluent. As used herein, “diluent” includes materials in which the particulate material can be dispersed, dissolved, or otherwise incorporated. Non-limiting examples of hydrophilic diluents are water, organic hydrophilic diluents such as lower monovalent alcohols and low molecular weight glycols and polyols, including propylene glycol, polyethylene glycol (e.g., Molecular Weight 200-600 g/mole), polypropylene glycol (e.g., Molecular Weight 425-2025 g/mole), glycerin, glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is a preferred diluent. The composition preferably comprises from about 70% to about 80% of the hydrophilic diluent.

Solutions according to the subject invention typically include a dermatologically acceptable hydrophilic diluent. Solutions useful in the subject invention preferably contain from about 70% to about 80% of the hydrophilic diluent.

Preferred carriers comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may also comprise a gel network, such as described in “Application of Emulsion Stability Theories to Mobile and Semisolid Oil-in-Water Emulsions”, Cosmetics and Toiletries, vol. 101, November 1986, pp. 73-92, which is incorporated by reference herein. Preferred emulsions are further described below.

The topical compositions of the subject invention, including but not limited to lotions and creams, may comprise a dermatologically acceptable emollient. Such compositions preferably contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients is known and may be used herein. Sagarin, Cosmetics, Science and Technology, 2nd Edition, Vol. 1, pp. 32-43 (1972), incorporated herein by reference, contains numerous examples of materials suitable as an emollient.

Lotions and creams according to the present invention generally comprise a solution carrier system and one or more emollients. Lotions typically comprise from about 1% to about 20%, preferably from about 5% to about 10%, of emollient; from about 50% to about 90%, preferably from about 60% to about 80%, water. A cream typically comprises from about 5% to about 50%, preferably from about 10% to about 20%, of emollient; and from about 45% to about 85%, preferably from about 50% to about 75%, water.

Compositions of this invention useful for cleansing (“cleansers”) are formulated with a suitable carrier, e.g., as described above, and preferably contain one or more dermatologically acceptable surfactants in an amount which is safe and effective for cleansing. Preferred compositions contain from about 1% to about 80%, more preferably from about 5% to about 10%, of a dermatologically acceptable surfactant. The surfactant is suitably selected from anionic, cationic, nonionic, zwitterionic, amphoteric and ampholytic surfactants, as well as mixtures of these surfactants. Such surfactants are well known to those skilled in the detergency art. Non-limiting examples of possible surfactants include isoceteth-20, sodium methyl cocoyl taurate, sodium methyl oleoyl taurate, sodium lauryl sulfate, and betaines such as described herein. See U.S. Pat. No. 4,800,197, to Kowcz et al., issued Jan. 24, 1989, which is incorporated herein by reference in its entirety, for exemplary surfactants useful herein. Examples of a broad variety of additional surfactants useful herein are described in McCutcheon's Detergents and Emulsifiers, North American Edition (1986), published by Allured Publishing Corporation, which is incorporated herein by reference in its entirety. The cleansing compositions can optionally contain, at their art-established levels, other materials which are conventionally used in cleansing compositions.

The physical form of the cleansing compositions is not critical. The compositions can be, for example, formulated as toilet bars, liquids, shampoos, bath gels, hair conditioners, hair tonics, pastes, or mousses. Toilet bars are most preferred since this is the form of cleansing agent most commonly used to wash the skin. Preferred rinse-off cleansing compositions, such as shampoos, include a delivery system adequate to deposit sufficient levels of actives on the skin and scalp. A preferred delivery system involves the use of insoluble complexes. For a more complete disclosure of such delivery systems, see U.S. Pat. No. 4,835,148, Barford et al., issued May 30, 1989, incorporated herein by reference in its entirety.

The compositions of the present invention are preferably formulated to have a pH of about 8 or below. The pH values of these compositions preferably range from about 2 to about 8, more preferably from about 3 to about 8, even more preferably from about 5 to about 7. In addition to the ingredients described above, the compositions, systems, and products of this invention can contain additional active and inactive ingredients or materials. Examples of these ingredients and materials include those known in that art, such as those described in Thomas et al, J. Am. Acad. Dermatol., vol. 4, No. 5, pp. 505-513 (5-1981), Kligman, A. M. & Willis, I. “A New Formula for Depigmenting Human Skin”, Archives of Dermatology, 111:40-48 (January 1975), Kligman A. M., Plewig, G. & Mills, O. H., “Topically Applied Tretinoin for Senile (Solar) Comedones”, Archives of Dermatology, 104: 420-421 (October 1971), Robinson T. A. & Kilgman, A. M., “Treatment of Solar Keratoses of the Extremities with Retinoic Acid and 5-Fluorouracil”, British Journal of Dermatology, 92:703-705 (1975), U.S. Pat. Nos. 2,716,981A, 2,728,339A, 2,882,892A, 2,923,291A, 3,499,446A, 3,949,741A, 4,117,837A, 4,585,797A, 4,591,501A, 4,631,227A, 4,748,976A, 4,942,029A, 5,026,552A, U.S. D355489S, U.S. Pat. Nos. 5,623,733A, 5,785,978A, 5,968,533A, 6,042,844A, 6,221,382B1, 5,723,138A, 5,869,072A, 5,997,887A, 5,961,479A, U.S. D426019S, U.S. Pat. Nos. 6,419,935B1, 6,548,075B1, 6,241,998B1, 6,316,021B1, US20030113356A1, US20050013784A1, U.S. Pat. Nos. 7,419,677B2, 6,649,181B1, US20040018166A1, U.S. Pat. No. 7,531,185B2, US20050238698A1, U.S. Pat. No. 7,514,071B2, US20060104931A1, US20090100558A1, ROC152926, US20090255554A1, U.S. Pat. Nos. 8,540,445B2, 8,387,163B2, US20110239347A1, U.S. Pat. No. 9,161,958B2, WO2012024403A2, US20120211022A1, WO2012153336A3, US20140150164A1, U.S. Pat. No. 9,032,554B2, EP2676703A2, US20150150326A1, U.S. Pat. No. 9,161,606B1, WO2016076443A1, U.S. Pat. No. 6,214,362B1, US20040242097A1, U.S. Pat. No. 6,419,935B1, US20040063603A1, US20080035174A1, US20030113356A1, WO1994002674A1, U.S. Pat. No. 6,957,924B1, US20030206940A1, US20030228351A1, U.S. Pat. No. 6,306,412B1, WO2004058214A1, WO2001008658A1, JP2003093152A, WO1997032567A1, US20070134304A1, WO2001078678A1, U.S. Pat. No. 6,887,486B2, US20080241200A1, US20080104787A1, US20040076660A1, US20030228352A1, WO2001002478A1, EP1813167A1, and U.S. Pat. No. 7,581,273B2.

Preferred topical compositions of the present invention comprise an emulsion. The purpose of this invention is to moderate and retard the aging changes in the skin by topical application of Vitamins A, C and E (or the derivatives or salts thereof) combination beginning in middle age when aging changes first become evident clinically. Certain of the anatomic alterations can be corrected and at least partially reversed, accompanied by improvement in the appearance of the skin.

The invention accomplishes two goals. First, a prophylactic effect in preventing progression and worsening of the damage with the passage of time. Secondly, various abnormalities are corrected and modified to the extent that the structure and function of the skin acquires the characteristics of younger skin.

Beneficial Effects of Vitamins A, C, and E Combination in Accordance with the Present Invention

### (a) Increases Proliferative Activity of Epidermal Cells

This results in thickening of the epidermis with correction of atrophy. Cell renewal is quickened so that cells divide at a rate typical of younger skin. Treatment with Vitamins A, C and E combination in accordance with the invention can double the skin thickness. The stimulation of cell growth also results in faster wound healing. Experiments have been performed wherein blisters have been raised and cut off on skins of individuals of various ages. Healing takes place in 2 or 3 weeks in young people, but takes much longer in older persons. Application of Vitamins A, C and E combination before raising the blister results in healing twice as fast in the older subjects.

### (b) Corrects Abnormalities of Differentiation

The Vitamins A, C and E combination disclosed herein regulates and controls the physiologic behavior of epithelial tissue, assuring its stability and integrity. It corrects and normalizes abnormalities of differentiation. In sun-damaged skin, the numerous foci of abnormal growths and segments of atypical, abnormal epidermis are corrected, reversed or eliminated. Fewer growths appear and progression to cancer is halted. Normalizing of the epidermis results in a smoother, less dry and rough skin, since cells are not only produced more rapidly but exfoliation occurs by individual cells rather than clusters or scales, thus improving the topography of the skin. Moreover, hyperpigmentation resulting in blotches and splotches is reduced by Vitamins A, C and E combination stopping excessive production of pigment by the melanocytes, although it cannot eliminate depigmentation.

### (c) The Metabolism of Fibroblasts is Increased

Fibroblasts synthesize the fibers of the dermis; new collagen is laid down, strengthening the physical foundation of the skin. Fibroblasts also make the ground substance which exists between the fibers, allowing these to glide past each other. The ground substance, known as acid mucopolysaccharides, is also responsible for the turgor and bounce of the skin. The Vitamins A, C and E combination disclosed herein stimulates the formation of new acid mucopolysaccharides.

Accordingly, the combination promotes the formation of a more normal dermis. Because of this activity, it has been found to promote and accelerate the healing of wounds in compromised tissue, of which regressed, aged dermis is an example. Further, the production of a new collagen layer not only repairs damaged skin but results in the effacement and prevention of fine wrinkles and lines.

### (d) Vascularity is Increased

The Vitamins A/C/E combination disclosed herein stimulates blood flow and promotes the formation of new vessels. Blood flow is greatly reduced in aged, sun-damaged skin. A brisker blood supply improves the physiologic competence of the skin and imparts a livelier, glowing appearance.

Several short term treatments using Vitamins A, C and E have claimed there is an increase in the blood flow in the skin. However, the increased blood flow from such short term treatments could result simply from vasodilation caused by the irritating effects of high concentrations of Vitamins A, C and E combination. In contrast, the low sub-irritating concentrations of Vitamins A, C and E combination according to the present invention do not cause significant vasodilation, but it has been found that over the long term there is not only a proliferation of new blood vessels, but also an increase in lymphocytes and other blood cells. As a result, there are more cells to fight infection, and the increased blood supply allows the skin to clear irritants and toxins more quickly from the skin.

Still further, treatment with Vitamins A, C and E combination according to the present invention raises the surface temperature of the skin by about ½ degree centigrade due to the greater basodermal flow of blood. The increased blood flow also increases acuity to pain and irritation, and the skin becomes more reactive to chemical insults. For example, experiments with highly drying and irritating cosmetics, soaps, perfumes, etc. have shown that young people will experience severe irritation within 3 or 4 days whereas it may take 2 or 3 weeks for an older person to feel the same irritation. The increased sensitivity of the skin treated with Vitamins A, C and E combination provides an early warning system to older people so that too much damage is not done before the pain or irritation is felt.

Vitamins A, C and E combination may be formulated in bland, moisterizing bases, such as creams or ointments, usually in the concentration range of about 0.005% to 0.05% and preferably about 0.01% to 0.025% by weight of base, although higher concentrations may be used for darker skins. Other non-toxic, dermatologically acceptable vehicles or carriers in which Vitamins A, C and E combination is stable will be evident to those of ordinary skill in the art. In general, emollient or lubricating vehicles, such as oleaginous substances, which help hydrate the skin are preferred. Volatile vehicles which dry or otherwise harm the skin, such as alcohol and acetone, should be avoided.

Vitamins A, C and E combination is a mild irritant and may cause redness and scaling, which may be accompanied by some tenderness and tightness. These reactions quickly disappear when the applications are stopped. Selection of an appropriate emollient vehicle will more readily allow the use of a highly effective but sub-irritating dose of the Vitamins A, C and E combination.

The extent or length of treatment according to the present invention may best be described as persistent or indefinite. That is, compared to the short term prior art treatments of various conditions with Vitamins A, C and E combination in which the treatments are terminated as soon as the condition disappears or subsides, the treatment according to the present invention is intended to continue indefinitely, otherwise the effects of aging will reappear after treatment is terminated. That is, the treatments of the present invention may be considered to be intervention therapy in decelerating the aging process. If the intervention is stopped, there is regression to the original state.

Usually, there is little point in beginning the treatments of the present invention until middle age when the effects of aging begin to appear. The particular program of maintenance therapy according to the present invention will vary depending upon the individual being treated. Generally, depending upon the age and state of the skin when treatments begin, it has been found that once every other day applications of Vitamins A, C and E combination for up to 6 months may be necessary to reduce and control the effects of aging which have already occurred. Once a stabilized skin control has been obtained, the frequency of application of Vitamins A, C and E combination may be reduced, for example to two times a week, and in some cases only once a week for the rest of the person's life. That is, once the aging process has been controlled, a maintenance dose on the order of two applications per week is generally sufficient to maintain that state.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

### Example 1

A stable topical composition was prepared from the following ingredients using conventional formulating techniques.

Example 1

| No. | Ingredients | % |
|---|---|---|
| 1 | Pure water | 79.31 |
| 2 | Sodium Ascorbyl Phosphate | 5.000 |
| 3 | Glycerin | 4.000 |
| 4 | Vitamin B3 | 4.000 |
| 5 | Sodium Lactate | 3.000 |
| 6 | Chondrus Crispus Extract | 1.000 |
| 7 | Hydrolyzed Jojoba Esters | 1.000 |
| 8 | Hydroxy Ethyl Cellulose | 0.600 |
| 9 | Panthenol | 0.500 |
| 10 | Cyclopeptide-5 | 0.500 |
| 11 | Phenoxyethanol | 0.450 |
| 12 | Sodium Hyaluronate | 0.080 |
| 13 | Lactic Acid | 0.080 |
| 14 | Ethylhexylglycerin | 0.050 |
| 15 | Tocopheryl Acetate | 0.040 |
| 16 | Retinol | 0.075 |

### Formulation Procedure:

Phase A: Dissolve 0.6 Gm of hydroxyl Ethyl Cellulose in an adequate amount of purified water. Phase B: Dissolve 4 Gm of Vitamin B3 in an adequate amount of pure water. Phase C: Dissolve 0.08 Gm of Sodium Hyaluronate in an adequate amount of pure water. Phase D: Dissolve 5 Gm of sodium Ascorbyl Phosphate in an adequate amount of Pure Water.
1). First, Phase A ingredients are combined and sparged using nitrogen for approximately 15 minutes, add 3 Gm of sodium Lactate into Phase A.
2). Phase B ingredients are then dissolved into Phase A until uniform using propeller type mixing and heated the mixture to about 75° C., then add 4 Gm of glycerin into the mixtures of Phase A/B. The mixture of phases A/B are then blanketed with a slow, steady stream of nitrogen.
3). In a separate vessel, Phase C ingredients are combined and heated to about 75° C. Next the Phase C ingredients are homogenized into the mixture of Phases A/B using any rotor/stator type of homogenizer for approximately 15 minutes. After the 15 minutes, the mixing is switched to low rpm sweep mixing. Then add 1 Gm of chondrus crispus extract, 1 Gm of hydrolyzed jojoba esters, 0.5 Gm of Panthenol, and 0.5 Gm of Cyclopeptide-5, into the Phase A/B/C and stir until complete solution.
4). Add Phase D into the above mixture of Phase A/B/C, then add 0.45 Gm of Phenoxyethanol, and 0.05 Gm of Ethylhexylglycerin. Once Phase A/B/C/D is mixed and the batch mixture is homogeneous, the entire batch mixture is cooled. When the batch is cooled to about 50° C., 0.08 Gm of lactic Acid is added to adjust pH to ˜6-7 and homogenized. When the batch is cooled to about 40° C., 0.04 Gm of Tocopheryl Acetate, and 0.75 Gm of Retinol are added to the batch mixture. Lastly, the batch mixture is cooled to about 30° C., and mixing is continued until the batch mixture is uniform.
The resulting composition is useful for application to the skin for delivering the retinyl palmitate and to treat and improve the appearance of the skin.

### Example 2

A stable topical composition was prepared from the following ingredients using conventional formulating techniques.

Example 2

| No. | Ingredients | % |
|---|---|---|
| 1 | Pure water | 77.31 |
| 2 | Sodium Ascorbyl Phosphate | 7.000 |
| 3 | Glycerin | 4.000 |
| 4 | Vitamin B3 | 4.000 |
| 5 | Sodium Lactate | 3.000 |
| 6 | Chondrus Crispus Extract | 1.000 |
| 7 | Hydrolyzed Jojoba Esters | 1.000 |
| 8 | Hydroxy Ethyl Cellulose | 0.600 |
| 9 | Panthenol | 0.500 |
| 10 | Cyclopeptide-5 | 0.500 |
| 11 | Phenoxyethanol | 0.450 |
| 12 | Sodium Hyaluronate | 0.080 |
| 13 | Lactic Acid | 0.080 |
| 14 | Ethylhexylglycerin | 0.050 |
| 15 | Tocopheryl Acetate | 0.040 |
| 16 | Retinyl Palmitate | 0.075 |

### Formulation Procedure:

Phase A: Dissolve 0.6 Gm of hydroxyl Ethyl Cellulose in an adequate amount of purified water. Phase B: Dissolve 4 Gm of Vitamin B3 in an adequate amount of pure water. Phase C: Dissolve 0.08 Gm of Sodium Hyaluronate in an adequate amount of pure water. Phase D: Dissolve 7 Gm of sodium Ascorbyl Phosphate in an adequate amount of Pure Water.
1). First, Phase A ingredients are combined and sparged using nitrogen for approximately 15 minutes, add 3 Gm of sodium Lactate into Phase A.
2). Phase B ingredients are then dissolved into Phase A until uniform using propeller type mixing and heated the mixture to about 75° C., then add 4 Gm of glycerin into the mixtures of Phase A/B. The mixture of phases A/B are then blanketed with a slow, steady stream of nitrogen.
3). In a separate vessel, Phase C ingredients are combined and heated to about 75° C. Next the Phase C ingredients are homogenized into the mixture of Phases A/B using any rotor/stator type of homogenizer for approximately 15 minutes. After the 15 minutes, the mixing is switched to low rpm sweep mixing. Then add 1 Gm of chondrus crispus extract, 1 Gm of hydrolyzed jojoba esters, 0.5 Gm of Panthenol, and 0.5 Gm of Cyclopeptide-5, into the Phase A/B/C and stir until complete solution.
4). Add Phase D into the above mixture of Phase A/B/C, then add 0.45 Gm of Phenoxyethanol, and 0.05 Gm of Ethylhexylglycerin. Once Phase A/B/C/D is mixed and the batch mixture is homogeneous, the entire batch mixture is cooled. When the batch is cooled to about 50° C., 0.08 Gm of lactic Acid is added to adjust pH to ˜6-7 and homogenized. When the batch is cooled to about 40° C., 0.04 Gm of Tocopheryl Acetate, and 0.75 Gm of Retinyl palmitate are added to the batch mixture. Lastly, the batch mixture is cooled to about 30° C., and mixing is continued until the batch mixture is uniform.

### Example 3

A stable topical composition was prepared from the following ingredients using conventional formulating techniques.

Example 3

| No. | Ingredients | % |
|---|---|---|
| 1 | Pure water | 79.660 |
| 2 | Sodium Ascorbyl Phosphate | 5.000 |
| 3 | Glycerin | 4.000 |
| 4 | Vitamin B3 | 4.000 |
| 5 | Sodium Lactate | 3.000 |
| 6 | Chondrus Crispus Extract | 1.000 |
| 7 | Hydrolyzed Jojoba Esters | 1.000 |
| 8 | Hydroxy Ethyl Cellulose | 0.600 |
| 9 | Panthenol | 0.500 |
| 10 | Cyclopeptide-5 | 0.500 |
| 11 | Phenoxyethanol | 0.450 |
| 12 | Sodium Hyaluronate | 0.080 |
| 13 | Lactic Acid | 0.080 |
| 14 | Ethylhexylglycerin | 0.050 |
| 15 | Tocopheryl Acetate | 0.040 |
| 16 | Retinyl Palmitate | 0.040 |

### Formulation Procedure:

Phase A: Dissolve 0.6 Gm of hydroxyl Ethyl Cellulose in an adequate amount of purified water. Phase B: Dissolve 4 Gm of Vitamin B3 in an adequate amount of pure water. Phase C: Dissolve 0.08 Gm of Sodium Hyaluronate in an adequate amount of pure water. Phase D: Dissolve 5 Gm of sodium Ascorbyl Phosphate in an adequate amount of Pure Water.
1). First, Phase A ingredients are combined and sparged using nitrogen for approximately 15 minutes, add 3 Gm of sodium Lactate into Phase A.
2). Phase B ingredients are then dissolved into Phase A until uniform using propeller type mixing and heated the mixture to about 75° C., then add 4 Gm of glycerin into the mixtures of Phase A/B. The mixture of phases A/B are then blanketed with a slow, steady stream of nitrogen.
3). In a separate vessel, Phase C ingredients are combined and heated to about 75° C. Next the Phase C ingredients are homogenized into the mixture of Phases A/B using any rotor/stator type of homogenizer for approximately 15 minutes. After the 15 minutes, the mixing is switched to low rpm sweep mixing. Then add 1 Gm of chondrus crispus extract, 1 Gm of hydrolyzed jojoba esters, 0.5 Gm of Panthenol, and 0.5 Gm of Cyclopeptide-5, into the Phase A/B/C and stir until complete solution.
4). Add Phase D into the above mixture of Phase A/B/C, then add 0.45 Gm of Phenoxyethanol, and 0.05 Gm of Ethylhexylglycerin. Once Phase A/B/C/D is mixed and the batch mixture is homogeneous, the entire batch mixture is cooled. When the batch is cooled to about 50° C., 0.08 Gm of lactic Acid is added to adjust pH to ˜6-7 and homogenized. When the batch is cooled to about 40° C., 0.04 Gm of Tocopheryl Acetate, and 0.04 Gm of Retinyl palmitate are added to the batch mixture. Lastly, the batch mixture is cooled to about 30° C., and mixing is continued until the batch mixture is uniform.

The resulting composition is useful for application to the skin for delivering the retinyl palmitate to treat and improve the appearance of the skin.

### Example 4

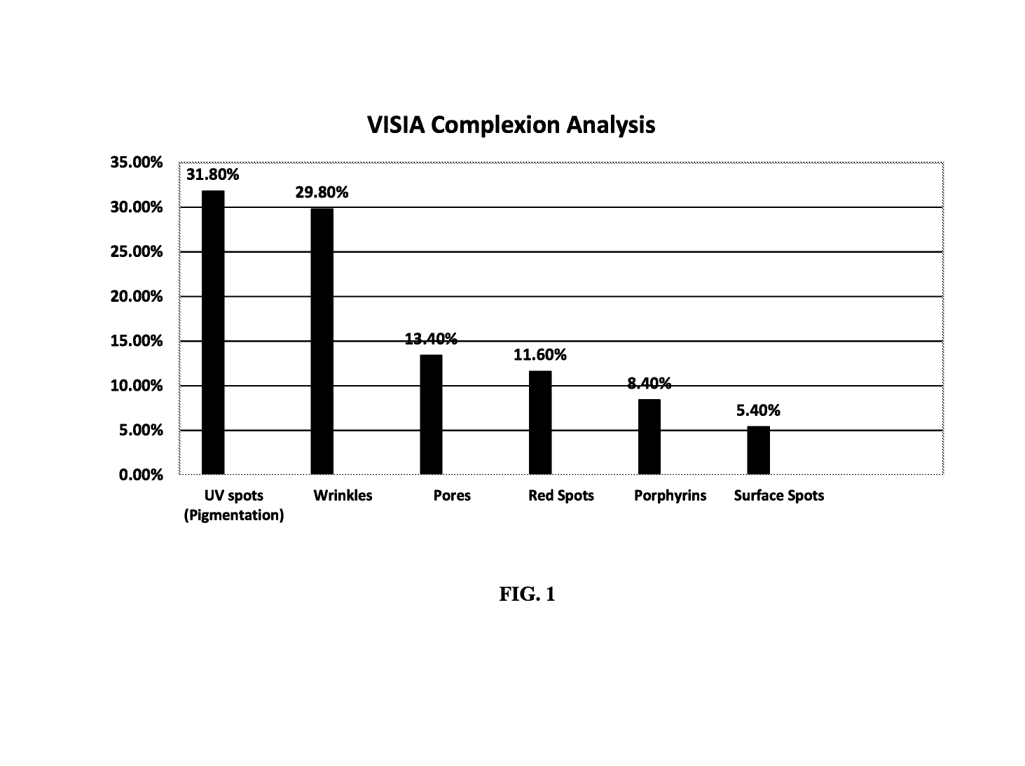

A formulation containing 0.04%, 5% and 0.04% by weight concentrations of Vitamins A, C and E was impregnated onto a pure silk facial membrane. The silk facial membrane was then applied to the faces of eight young and middle-aged women. They ranged in age from 23 to 50. Briefly, the facial membrane applied every night before bedtime for 15 minutes for two week. Clinical assessments were made every week. The results were shown in FIG. 1.

As shown in FIG. 1, the average rate of improvement of de-pigmentation is estimated around 31.8%, of anti-wrinkle effect is 29.80%, pores size reduction by 13.4%, reduction of inflammation and comedones by 11.6% and 8.4%. The cosmetic beneficial effects were obtained in about 80% after about two weeks. Improvement was maximal at about four months and persisted as long as the membrane was used. Withdrawal of the membrane resulted in a slow loss of improvement with a return to the original state in about four to five months. Maintenance therapy was required to prevent relapse. Most of those who improved continued to enjoy the use Vitamins A, C and E combination once every other day. The beneficial effects included effacement of small wrinkles, smoother surface, pigmentation greater turgor, elimination of actinic keratoes, elimination of senile comedones, less conspicuous pores and less mottling (fading of pigmented spots). At least over 100 persons have used typical Vitamins A, C and E combination experimentally.

From the foregoing, it was found that the invention has the following advantages and improvements inter alia:

### A. Clinical

Clinical improvement included effacement of fine wrinkles, smoother surface, and lightens pigmented blotches. Additionally, it was found that skin has more turgor, large pores were less noticeable, and skin felt livelier

### B. Histologic

At histologic level, the following improvements were found: thicker epidermis, normalizes atypia and pre-malignant changes, and atrophy and dysplasia corrected. In addition, it was found that the formulation stimulated blood flow and new vessels formed. The formulation also stimulated fibroblasts with new collagen formation and increased ground substance. Melanin within keratinocytes was also decreased.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A skin care formulation comprising:
from about 0.0005% to about 1% by weight of retinol, retinal or retinyl palmitate;
from about 1% to about 15% by weight of Vitamin C or a derivative thereof;
from about 0.005% to about 0.5% by weight of Vitamin E or a derivative thereof; and
a dermatologically or cosmetically acceptable carrier.

2. The formulation according to claim 1, further comprising one or more of the following:
from about 70% to about 80% by weight of pure water;
from about 0.05% to about 8% of moisturizing agent comprising one or more selected from the group consisting of Glycerin, Sodium Hyaluronate, Snail extract, Mannitol, Panthenol, Cyclopeptide, Ammonium Glycyrrhizate, and a combination thereof;
from about 1% to about 3% or a derivative thereof of a structuring agent selected from the group consisting of Xanthan Gum and Chondrus Crispus Extract, Hydroxy Ethyl Cellulose, and a combination thereof;
from about 0.25% to about 2% by weight of a buffer/pH adjusting agent selected from the group consisting of citric acid, lactic acid, and a combination thereof; and
from about 0.1% to about 0.5% by weight of a preservative selected from group consisting of phenoxyethnol, ethylhexlglycerin, methyl/propyl paraben, and a combination thereof.

3. The formulation according to claim 1, wherein said formulation further comprises a compound selected from the group consisting of hydroxy acids, desquamatory agents, sunscreens, anti-oxidants, and combinations thereof.

4. The formulation according to claim 1, wherein the carrier is in the form of essence liquid.

5. The formulation according to claim 1, wherein the formulation is the form of a cream or lotion.

6. A delivery system comprising (i) a formulation according to claim 1, and (ii) a substrate, wherein the formulation is impregnated in or on the substrate.

7. The delivery system of claim 6, wherein the substrate comprises a pure gentle mixing double-sided knitting silk membrane.

8. The delivery system of claim 6, wherein the delivery system is a facial mask.

9. A method of regulating skin condition, comprising applying to a surface of skin in need thereof a formulation according to claim 1 for a period of time.

10. A method of regulating visible and/or tactile discontinuities in the texture of mammalian skin, comprising applying to a surface of skin in need thereof a formulation according to claim 1.

11. The method according to claim 9, wherein said skin is human facial skin.

12. The method according to claim 9, wherein the period of time is 1 minute to 24 hours, 5 minutes to 12 hours, 10 minutes to 8 hours, or 15 minutes to 1 hour.
